(19) United States
(12) Patent Application Publication
(10) Pub. No.: US 2016/0220544 A1
(43) Pub. Date: Aug. 4, 2016

(54) PHARMACEUTICAL COMPOSITION OF SODIUM PICOSULFATE

(71) Applicant: Gavis Pharmaceuticals, Somerset, NJ (US)

(72) Inventors: Ganesh Kumar Palaniappan Appavu, Somerset, NJ (US); Arjun Yadhav Muthukkaruppan, Somerset, NJ (US); James Garegnani, Somerset, NJ (US)

(73) Assignee: Gavis Pharmaceuticals, Somerset, NJ (US)

(21) Appl. No.: 14/611,237

(22) Filed: Jan. 31, 2015

(51) Int. Cl. A61K 31/4402; A61K 33/08; A61K 9/16; A61K 31/194

(52) U.S. Cl. CPC A61K 31/4402; A61K 31/194; A61K 33/08; A61K 9/1682; A61K 9/1688

(57) ABSTRACT

A pharmaceutical composition comprising granules which comprises sodium picosulfate, citric acid and magnesium oxide, prepared without using any solvent is provided. The composition ensures homogeneity or content uniformity of the ingredients as well as being prepared by a simple manufacturing process. Preferably, the granules in the composition are prepared by compacting and milling a mixture of sodium picosulfate, citric acid and magnesium oxide followed by mixing with other optional ingredients.

### BACKGROUND OF THE INVENTION

(a) Field of the Invention

The present invention is directed to a pharmaceutical composition of sodium picosulfate which is prepared by an improved manufacturing process. The composition comprises a physical mixture of sodium picosulfate, citric acid, magnesium oxide, and a bicarbonate along with other ingredients. The invention is further directed to the use of said composition for cleansing of the colon as a preparation of colonoscopy in adults.

(b) Description of the Related Art

A pharmaceutical product used for clearance of the bowel prior to X-ray examination, endoscopy or surgery, is presently sold under the trade mark name of Picolax™ The pharmaceutical product is a white powder which is made up as a solution (in water) for administration. The properties required are that it is a strong laxative that is easily palatable. The pharmaceutical product includes sodium picosulphate, a stimulant laxative; and anhydrous citric acid and magnesium oxide (MgO, light), which together in solution form magnesium citrate, an osmotic laxative with a powerful cathartic effect.

The dosage form for oral delivery is in the form of granules. Herein the term granule(s) includes loose particles (such as particles which might collectively be termed a powder, including loose particles in the form of a powder which is known in the art as “powder for oral administration”). The product is a physical mixture of six raw materials; these being citric acid (e.g. citric acid anhydrous or citric acid monohydrate), magnesium oxide (e.g. magnesium oxide light), potassium bicarbonate (KHCO₃), sodium picosulphate (NaPIC), sodium saccharin, and orange flavour. Magnesium oxide “light” means, herein, magnesium oxide having an apparent volume such that 15 g occupies between 75 to 180 ml, e.g. 15 g occupies a volume of 150 ml.

The known process for making Picolax™ may include the following steps. Granules of magnesium oxide and citric acid are produced by mixing the two reagents together; this mixture is known as the “primary mix”. In another stage, potassium bicarbonate, sodium picosulphate and water are mixed or blended to produce a wet “pre-mix”, which is then dried. In a further stage, the flavour ingredients, orange flavour and sodium saccharin, are blended with the pre-mix and primary mix. The known process has several associated problems.

Firstly, the mixing processes may result in inhomogeneity problems in the final and intermediate products. In one aspect, the terms “inhomogeneity” and “lack of homogeneity” as used in this application refer to the lack of uniformity of content of the active substance, sodium picosulphate, in the final product. It also refers to the lack of homogeneity in the physical and morphological properties, such as the particle size (diameter) or particle size range or distribution, of the intermediate products and/or the final product granules. Intermediate product granules are, for example, the primary mix granules or the pre-mix granules.

Homogeneity has been suspected to be at least one of the critical factors assuring the quality and performance of the final product, and it is believed that product homogeneity (and inhomogeneity) relates to the mixing processes used. Thus, in the first stage of the known process, disparities may occur in the granule size and distribution (i.e. inhomogeneity may arise) because of the low binding properties or agglomeration properties between citric acid and magnesium oxide particles (caused by e.g. the difference in densities of the two materials). Further, magnesium oxide is left on the mixer bowl, blades, etc. (rather than being mixed with the citric acid). Thus, in the known process, extra magnesium oxide (“overage”) is included in the raw materials to compensate for losses during the blending process. The overage is typically greater than 10%. This leads to economic losses over longer periods and where larger quantities are produced. Additionally, longer processing times are entailed, and unhealthy amounts of dust may be produced during mixing.

In the premix stage, lack of homogeneity of the resulting granules may arise due to dissolution of some potassium bicarbonate in the granulation medium, water, and because of physical degradation (smashing) of the particles during mixing. This may have a detrimental effect on the final product. Further, long processing times, and multiple steps, are required to complete this stage of the process (which takes typically 15 to 24 hours).

U.S. Pat. Nos. 8,450,338 and 8,481,083 disclose a composition of sodium picosulfate containing pre-mix granules having a spray coated layer of sodium picosulfate and solvent, e.g. water over the potassium bicarbonate core. The primary mix of magnesium oxide and citric acid was then added to the pre-mix granules to form the final product.

The process according to the patents, involving spray coating of aqueous sodium picosulfate solution over sodium bicarbonate, would still cause dissolution of the potassium bicarbonate during the coating process that may cause physical degradation (smashing) of the potassium bicarbonate particles during coating. As a result, this process, in line with the other known prior art processes, thus may not ensure homogeneity and content uniformity of the final product.

The patents further teach the need to use sophisticated mixing apparatus, e.g. multi-dimensional blender or three dimensional blender for mixing of magnesium oxide and citric acid in order to reduce magnesium oxide overages and homogeneity problem due to the density differences between the two ingredients.

Thus, there is a need for an alternate, improved and simple manufacturing process.

### SUMMARY OF THE INVENTION

The present invention provides a pharmaceutical composition and a process of manufacturing it which may alleviate some or all of the problems of the prior art process, e.g. use simple, improved manufacturing process and provide a product with desired content uniformity.

The composition according to the invention comprises granules, which comprise sodium picosulphate, citric acid and magnesium oxide. The granules are prepared without the use of any liquid solvent, e.g. water. Preferably, the mixture of sodium picosulphate, citric acid and magnesium oxide is compacted using a roller compactor and then milled to form granules of desired size.

In one aspect, the invention provides a pharmaceutical composition comprising granules which comprise sodium picosulphate, citric acid and magnesium oxide, wherein the granules are prepared without the use of a solvent, e.g., a liquid solvent.

In one aspect, the pharmaceutical composition consists essentially of sodium picosulphate, citric acid and magnesium oxide for cleansing the colon. The composition may further include a mixture of flavours, sweeteners, and a bicarbonate. The pharmaceutical composition may be characterized by being prepared without the use of a solvent.

In another aspect, the pharmaceutical composition may consist of sodium picosulphate, citric acid and magnesium oxide for cleansing the colon. In another aspect, the composition may consist of the sodium picosulphate, citric acid and magnesium oxide and a mixture of flavours, sweeteners, and a bicarbonate. The pharmaceutical composition may be characterized by being prepared without the use of a solvent.

In another aspect, the invention provides a pharmaceutical composition comprising:
(a) granules which comprise sodium picosulphate, citric acid and magnesium oxide; and
(b) mixture of flavours, sweeteners, and a bicarbonate,
   wherein said granules are prepared without the use of a solvent.

In another aspect, the granules in the composition are prepared by: (a) mixing sodium picosulfate, citric acid and magnesium oxide, (b) compacting the mixture; and (c) milling the compacted mixture to form granules.

In another aspect, the process of preparing the granules comprising sodium picosulphate, citric acid and magnesium oxide, is devoid of a step of wet granulation or spray coating. Preferably the granules are devoid of a spray coating layer or the composition is devoid of granules prepared by wet granulation or spray coating.

In another aspect, the process of preparing the granules comprising sodium picosulphate, citric acid and magnesium oxide is devoid of step of drying.

In another general aspect, the process of preparing the pharmaceutical composition comprises the steps of:
(a) mixing sodium picosulfate and citric acid; and
(b) mixing magnesium oxide with the mixture of step (a) to form granules,
   wherein the process does not involve use of any solvent, i.e., the process is free of a solvent.

In this general aspect, the process may consist essentially of the steps of (a) mixing sodium picosulfate and citric acid; and (b) mixing magnesium oxide with the mixture of step (a) to form granules. The process may be devoid of a step of wet granulation and/or spray coating. The process may further include the steps of mixing flavours, sweeteners and a bicarbonate with the granules.

In another general aspect, the process may consist of the steps of (a) mixing sodium picosulfate and citric acid; (b) mixing magnesium oxide with the mixture of step (a) to form granules; and (c) filling the granules into a sachet. The process may be devoid of a step of wet granulation and/or spray coating. The process may further include the steps of mixing flavours, sweeteners and a bicarbonate with the granules.

In another general aspect, the process of preparing the pharmaceutical composition further comprises steps of compaction of the mixture of step (a) and magnesium oxide followed by milling to form granules. Preferably, the process is devoid of a step of wet granulation and/or spray coating.

In another general aspect, the process may consist of or consist essentially of the steps of (a) mixing sodium picosulfate and citric acid; (b) mixing magnesium oxide with the mixture of step (a); (c) compacting the mixture to form granules; and (d) filling the granules into a sachet. The process may be devoid of a step of wet granulation and/or spray coating. The process may further include the steps of mixing flavours, sweeteners and a bicarbonate with the granules.

In another general aspect, the process of preparing the pharmaceutical composition further comprises steps of mixing flavours, sweeteners and a bicarbonate with the milled granules.

In another general aspect, the invention provides a process for the preparation of a pharmaceutical composition comprising a mixture of citric acid, sodium picosulfate, magnesium oxide, a bicarbonate, and optionally, a sweetener such as saccharine sodium and a flavour such as orange flavour, comprising:
(a) mixing sodium picosulfate and citric acid;
(b) mixing magnesium oxide with the mixture of step (a);
(c) compacting the mixture of step (b) followed by milling to form granules; and
(d) mixing bicarbonate and optionally, flavours and sweeteners with the granules,
   wherein the process does not involve use of any solvent.

In this general aspect, the process may consist essentially of or consist of the steps of (a) mixing sodium picosulfate and citric acid, (b) mixing magnesium oxide with the mixture of step (a) to form granules, (c) compacting the mixture of step (b) followed by milling to form granules, (d) mixing bicarbonate and optional flavours and sweeteners with the granules; and (e) filling the granules into a sachet. The process may be devoid of a step of wet granulation and/or spray coating.

In another general aspect, more than 85% of the granules of the pharmaceutical composition have a diameter between about 100 μm and about 900 μm.

In another general aspect, less than 5% of the granules of the pharmaceutical composition have a diameter greater than about 900 μm, or less than 5% of the granules have a diameter less than about 100 μm.

The pharmaceutical composition of the present invention may be used for clearance of the bowel prior to X-ray examination, endoscopy or surgery.

Still other aspects and advantages of the invention will be apparent from the following detailed description of the invention.

### DETAILED DESCRIPTION OF THE INVENTION

The invention provides for a pharmaceutical composition for cleansing of the colon as a preparation of colonoscopy in adults. The composition comprises a mixture of granules comprising sodium picosulfate, citric acid and magnesium oxide; and a bicarbonate, optionally flavours and sweeteners. The granules are not prepared by wet granulation or using any solvent system. Preferably, the mixture of sodium picosulfate, citric acid and magnesium oxide is subjected to compaction, e.g. roller compaction, milled and mixed with other ingredients including a bicarbonate and optionally, sweeteners and flavours.

The granules contain a homogeneous or substantially homogeneous mixture of the content, preferably sodium picosulfate, citric acid and magnesium oxide.

In the prior art process, disparities were found to occur in the granule size and distribution, apparently due to the low binding properties or agglomeration properties between the citric acid and magnesium oxide particles. The blending instruments used in the prior art, such as a tumble blender or a planetary dry mixer, appeared to encourage separation of the two components, and loss of raw material in the form of fines, for example, of magnesium oxide. As a result, using the known process, it was necessary to compensate on a regular basis for losses by adding extra magnesium oxide (“overage”) in an amount of typically above 10%, which leads to economic losses over longer periods and larger quantities produced. Additionally, long processing times may be entailed, and unhealthy amounts of magnesium oxide dust may be produced during mixing. The prior art process may result in cleaning difficulties, and/or poor control of product granule/particle size and distribution. The prior art further teaches one to use sophisticated blenders such as a multi-dimension blender or a three-dimensional blender for mixing magnesium oxide and citric acid which still may have the aforementioned issues due to density differences between the two ingredients.

It was found that by adopting the manufacturing process of the invention, which does not require mixing of magnesium oxide directly into citric acid as a single ingredient, the problems encountered in the prior process are either removed or significantly reduced. Advantageously, the process is simple, less time consuming, does not require adjustment of magnesium oxide overages or use of sophisticated mixing equipment.

Further, the prior art process requires either wet mixing/granulation or solvent based spray coating of the sodium picosulphate and bicarbonate to form pre-mix granules. The prior art process thus cause dissolution of part of the quantity of bicarbonate. As a result, there may be a loss of product homogeneity because either overly large particles or granules contain less sodium picosulphate and/or overly fine particles or granules of the dried mixture contain too much sodium picosulphate.

The process of the invention firstly, does not use any solvent system to form granules (i.e., not prepared by either wet granulation or spray coating), and secondly, bicarbonate is not granulated along with sodium picosulfate. The process thus ensures minimum loss of bicarbonate and as a result, the end product has the desired content uniformity.

The homogeneous or substantially homogeneous mixture of sodium picosulphate, citric acid and magnesium oxide may be in the form of granules.

Subsequently, the process of the invention involves mixing of saccharin sodium, orange flavour, part of the granules, and a bicarbonate selected from sodium or potassium bicarbonate to provide the final homogeneous bulk product.

The granule(s) may have a particle size (diameter) range or distribution of between about 100 and about 900 μm, e.g. between about 150 and 875 μm, e.g. between about 250 and about 850 μm. The pharmaceutical composition may be in the form of granules of, e.g. a particle size (diameter) range or distribution of between about 100 and about 900 μm, e.g. between about 150 and 875 μm, e.g. between about 250 and about 850 μm.

It will be appreciated that herein the term diameter is not intended to mean that any of the particles and granules disclosed are spherical. The granules may be, for example, roughly spherical, in the form of elongated spheres (ellipsoidal) etc. Herein the term size (diameter) is intended to mean the shortest distance in a straight line passing from one side to the other through the centre point of the granule (e.g. sphere, rough sphere, elongated sphere, ellipsoid).

According to the present invention, there is provided a pharmaceutical composition comprising a mixture of a granule or granules which comprises a homogeneous or substantially homogeneous mixture of sodium picosulphate, citric acid, and magnesium oxide; and a bicarbonate, and optionally, saccharin sodium and orange flavour. Preferably, the composition is devoid of granules prepared by wet granulation or spray coating.

The granule(s) may have a particle size (diameter) range or distribution of between about 100 and about 900 μm, e.g. between about 150 and 875 μm, e.g. between about 250 and about 850 μm.

The content uniformity of the active substance, sodium picosulphate, in the final product granule(s) or pharmaceutical composition may have a mean value of about 0.0559% and 0.068% by weight (9.0-11.0 mg/dose, based on a dose of 16.1 g Picolax™).

The mixture of granule(s) and other ingredients (bicarbonate, flavours and sweeteners) may be dispensed as sachets.

The invention further provides a process for the preparation of a pharmaceutical composition. The process comprises steps of:
(a) mixing sodium picosulfate and citric acid; and
(b) mixing magnesium oxide with the mixture of step (a) to form granules,
   wherein the process is free of the use of a solvent.

The process additionally comprises compaction of the mixture of step (b) followed by milling to form granules.

The process further may comprise mixing of bicarbonate, optionally, orange flavour and saccharin sodium with the milled granules.

In an embodiment, the process is devoid of step of wet granulation and/or spray coating. In another embodiment, the process is devoid of a step of drying.

In an embodiment, the process for the preparation of a pharmaceutical composition of citric acid, sodium picosulfate, magnesium oxide, and bicarbonate comprises the steps of:
(a) mixing sodium picosulfate and citric acid;
(b) mixing magnesium oxide with the mixture of step (a);
(c) compacting the mixture of step (b) followed by milling to form granules; and
(d) mixing bicarbonate and optionally, flavours and sweeteners with the granules.

In another embodiment, the process for the preparation of the pharmaceutical composition comprises the steps of:
(a) mixing magnesium oxide and citric acid followed by compacting and milling the mixture to form granules;
(b) mixing half quantity of the granules with sodium picosulfate;
(c) mixing remaining quantity of the granules with bicarbonate, sweetener and flavour; and
(d) mixing the mixtures prepared in step (b) and (c).

In another embodiment, the process for the preparation comprises the steps of:
(a) mixing sodium picosulfate, magnesium oxide and citric acid followed by compacting and milling the mixture to form granules; and
(b) mixing the granules with bicarbonate, sweetener and flavour.

In another embodiment, the process for the preparation of the pharmaceutical composition comprises the steps of:
(a) mixing magnesium oxide and a part quantity of citric acid followed by compacting and milling the mixture to form granules;
(b) mixing sodium picosulfate and the remaining quantity of citric acid;
(c) mixing the granules with the mixture prepared in step (b); and
(d) mixing bicarbonate, sweetener and flavour with the mixture prepared in step (c).

In another embodiment, the process for the preparation of the pharmaceutical composition comprises the steps of:
(a) mixing magnesium oxide and a part quantity of citric acid followed by compacting and milling the mixture to form granules;
(b) preparing a mixture of sodium picosulfate, the remaining quantity of citric acid, bicarbonate, sweetener, and flavour; and
(c) mixing the mixture prepared in step (b) with the granules.

In a further embodiment, the process for the preparation of pharmaceutical composition comprises steps of:
(a) mixing magnesium oxide and a part quantity of citric acid followed by compacting and milling the mixture to form granules;
(b) mixing the remaining quantity of citric acid with sodium picosulfate followed by compacting and milling the mixture to form granules;
(c) mixing the granules prepared in step (b) with bicarbonate, sweetener and flavour; and
(d) mixing the granules prepared in step (a) with the mixture prepared in step (c).

The process of the invention may include a separation (e.g. processing, e.g. milling or sieving) step or steps e.g. to obtain bicarbonate of appropriate size and/or size distribution e.g. a particle size (diameter) range of, for example, between about 100 and about 900 μm, e.g. between about 150 and 875 μm, e.g. between about 250 and about 850 μm prior to compressing. The process of the invention may include a separation (e.g. processing, e.g. sieving) step or steps e.g. to obtain citric acid or magnesium oxide of appropriate size and/or size distribution e.g. a particle size (diameter) range of, for example, between about 100 and about 900 μm, e.g. between about 150 and 875 μm, e.g. between about 250 and about 850 μm.

The process of preparing the granules in the composition of the invention is preferably a dry granulation method. In dry granulation, the ingredients are not exposed to moisture, solvents and heat. Dry granulation can be carried out by slugging or roller compaction. Slugging is a double compression process. The material to be tabletted is compressed to a large compressed mass, or “slug,” which is converted to tablets by a second compression process. Because slugging is a slow and uneconomic process, roller compaction has become the method of choice for dry granulation. Roller compaction has all the benefits of a granulation process, such as improved material flow behaviour and content uniformity. In addition, roller compaction is high-volume and more economical to operate.

In the roller compaction process (also known in the art as “roll compaction”), a roller compactor uses pressure to compact and densify the ingredients and to bind powders into granules.

Granulation is a process of size enlargement in which small particles are gathered together into larger aggregates in which the original particles can still be identified. Uniformly mixed powders (granulate formulations) are compressed between counter rotating rollers to form a ribbon of compacted material that is then milled into granules. A roller compactor comprises a roller assembly, press frame, hydraulic pressure system, and a feed system. The feed system is located immediately before the rollers and determines the rate of flow of the granulate formulation to the rollers. The feed system may comprise one or more feed screws that force the granulate formulation between the compacting rollers. The granulate formulation is compacted as it passes through the two compacting rollers. The volume of the granulate formulation decreases as it passes through the region of maximum pressure, where it is formed into a solid compacted material known as a sheet or ribbon. Compaction pressure is provided by the hydraulic pressure system, which can be adjusted to produce the desired compaction pressure. The hydraulic pressure system acts on one of the rollers. The roller compaction process may be a continuous process of compacting, milling, screening, and recycling the too large granules (also known as “Overs”) and too small granules (also known as “Fines”) back to the process.

Various configurations for the rollers are well known in the art and are described, for example, in A. M. Falzone, Ph.D. Thesis, Purdue University, 1990 (U.M.I., Ann Arbor, Mich., Order Number 9313940). Roller compaction equipment is commercially available from the Fitzpatrick Company, Elmhurst III. USA as CHILSONATOR® roll compactors. This equipment is described in “Introduction to Roll Compaction and the Fitzpatrick CHILSONATOR,” published by The Fitzpatrick Company Europe.

The invention further provides use of the pharmaceutical composition as substantially disclosed herein for clearance of the bowel prior to X-ray examination, endoscopy or surgery.

### Example 1

### Sodium Picosulfate, Magnesium Oxide, Anhydrous Citric Acid (10 mg, 3.5 g, 12 g) Powder for Oral Solution

TABLE 1

| Sr. No. | Ingredient | Quantity (mg/Pouch) |
|---|---|---|
| 1 | Sodium Picosulfate | 10 |
| 2 | Magnesium Oxide | 3500 |
| 3 | Citric Acid Anhydrous | 12,000 |
| 4 | Potassium Bicarbonate | 500 |
| 5 | Sodium Saccharin | 50 |
| 6 | Orange Flavour | 40 |
| | Total | 16,100 |

### Process:

Magnesium Oxide and Citric Acid Anhydrous were screened, mixed and blended. The mixture was subjected to roller compaction followed by milling to form granules. A half quantity of the resulting milled granules were blended with Sodium Picosulfate. The remaining quantity of the milled granules was blended with Potassium Bicarbonate, Sodium Saccharin, and Orange Flavour. The final mixture was then filled into pouches.

### Ingredient Specifications:

(A) Input Sodium Picosulfate PSD: 100% of the particles less than 30 μm.

### (B) Input Citric Acid PSD: 250 μm to 600 μm.

(C) Final Blend PSD: 50% of final blend particles are less than 75 μm and 50% of final blend particles are between 100 μm to 400 μm.

### Example 2

### Sodium Picosulfate, Magnesium Oxide, Anhydrous Citric Acid (10 mg, 3.5 g, 12 g) Powder for Oral Solution

The formulation summarized in Table 1 of Example 1 was prepared by the following process:

Sodium Picosulfate, Magnesium Oxide and Citric Acid Anhydrous were screened, mixed and blended. The mixture was subjected to roller compaction followed by milling to form granules. The milled granules were blended with Potassium Bicarbonate, Sodium Saccharin, and Orange Flavour. The final mixture was then filled into pouches.

### Example 3

### Sodium Picosulfate, Magnesium Oxide, Anhydrous Citric Acid (10 mg, 3.5 g, 12 g) Powder for Oral Solution

The formulation summarized in Table 1 of Example 1 was prepared by the following process:

Magnesium Oxide and Citric Acid Anhydrous were screened, mixed and blended. Sodium Picosulfate was then added and blended with the mixture. The mixture was subjected to roller compaction followed by milling to form granules. The milled granules were blended with Potassium Bicarbonate, Sodium Saccharin, and Orange Flavour. The final mixture was then filled into pouches.

### Example 4

### Sodium Picosulfate, Magnesium Oxide, Anhydrous Citric Acid (10 mg, 3.5 g, 12 g) Powder for Oral Solution

The formulation summarized in Table 1 of Example 1 was prepared by the following process:

Magnesium Oxide and 11,900 mg or 10,000 mg of Citric Acid Anhydrous were screened, mixed and blended. The mixture was subjected to roller compaction followed by milling to form granules. Separately, Sodium picosulfate and 100 mg or 2000 mg of Citric Acid Anhydrous were screened, mixed and blended. The mixture was then milled and mixed with milled granules such that the mixture had 12,000 mg of citric acid anhydrous present. The blend was further added with Potassium Bicarbonate, Sodium Saccharin and Orange Flavour. The final mixture was then filled into pouches.

### Example 5

### Sodium Picosulfate, Magnesium Oxide, Anhydrous Citric Acid (10 mg, 3.5 g, 12 g) Powder for Oral Solution

The formulation summarized in Table 1 of Example 1 was prepared by the following process:

Magnesium Oxide and 11,990 mg or 10,000 mg of Citric Acid Anhydrous were screened, mixed and blended. The mixture was subjected to roller compaction followed by milling to form granules. Separately, 10 mg or 2,000 mg of Citric Acid Anhydrous were screened and milled followed by addition of Sodium picosulfate. Potassium Bicarbonate, Sodium Saccharin and Orange Flavour were then added to the mixture. Finally milled granules were added to the said mixture such that the resulting mixture had 12,000 mg of citric acid anhydrous. The mixture was then filled into pouches.

### Example 6

### Sodium Picosulfate, Magnesium Oxide, Anhydrous Citric Acid (10 mg, 3.5 g, 12 g) Powder for Oral Solution

The formulation summarized in Table 1 of Example 1 was prepared by the following process:

Magnesium Oxide and 11,990 mg or 10,000 mg of Citric Acid Anhydrous were screened, mixed and blended. The mixture was subjected to roller compaction followed by milling to form granules. Separately, 10 mg or 2,000 mg of Citric Acid Anhydrous, Sodium picosulfate and Sodium Saccharin were screened, mixed and milled. Potassium Bicarbonate, Sodium Saccharin and Orange Flavour were then added to the mixture. Finally milled granules were added to the mixture such that the resulting mixture had 12,000 mg of citric acid anhydrous, and the mixture was then filled into pouches.

### Example 7

### Sodium Picosulfate, Magnesium Oxide, Anhydrous Citric Acid (10 mg, 3.5 g, 12 g) Powder for Oral Solution

The formulation summarized in Table 1 of Example 1 was prepared by the following process:

(1) Magnesium Oxide and 11,990 mg or 10,000 mg of Citric Acid Anhydrous were screened, mixed and blended. The mixture was subjected to roller compaction followed by milling to form granules.
(2) Separately, 10 mg or 2,000 mg of Citric Acid Anhydrous were screened and milled followed by addition of Sodium picosulfate. The mixture was subjected to roller compaction followed by milling to form granules. Potassium Bicarbonate, Sodium Saccharin and Orange Flavour were then added to the milled granules mixture.
(3) Finally milled granules prepared in step (1) and blend prepared in step (2) were mixed and the mixture was then filled in pouches such that the resulting mixture had 12,000 mg of citric acid anhydrous.

1. A pharmaceutical composition comprising granules which comprises sodium picosulphate, citric acid and magnesium oxide, wherein the granules are prepared without the use of a solvent.

2. The pharmaceutical composition of claim 1, wherein the granules are prepared in a process comprising compacting sodium picosulphate, citric acid and magnesium oxide together to form compacts and then milling the compacts.

3. The pharmaceutical composition of claim 1, wherein the composition is devoid of granules prepared by wet granulation or spray coating.

4. The pharmaceutical composition of claim 1, wherein the granules have a diameter between about 100 μm and about 900 μm.

5. The pharmaceutical composition of claim 1, wherein more than 85% of the granules have a diameter between about 100 μm and about 900 μm.

6. The pharmaceutical composition of claim 1, wherein less than 5% of the granules have a diameter greater than about 900 μm; or wherein less than 5% of the granules have a diameter less than about 100 μm.

7. The pharmaceutical composition of claim 1, further comprising one or more of flavours, sweeteners and a bicarbonate.

8. A process for the preparation of a pharmaceutical composition comprising granules which comprises sodium picosulphate, citric acid and magnesium oxide, wherein the process comprises the steps of:
(a) mixing sodium picosulfate and citric acid; and
(b) mixing magnesium oxide with the mixture of step (a) to form granules,
wherein the granules are prepared without the use of a solvent.

9. The process of claim 8, further comprising compacting the mixture of step (b) followed by milling to form granules.

10. The process of claim 8, wherein the process is devoid of a step of wet granulation and/or spray coating.

11. The process of claim 8, wherein the process is devoid of a step of drying.

12. The process of claim 8, further comprising mixing one or more of flavours, sweeteners and a bicarbonate with the granules.

13. The process of claim 12, wherein the bicarbonate is preheated prior to mixing with the granules.

14. The process of claim 8, wherein the granules have a particle diameter between about 100 μm and about 900 μm.

15. The process of claim 8, wherein more than 85% of the granules have a diameter between about 100 μm and about 900 μm.

16. The process of claim 8, wherein less than 5% of the granules have a diameter greater than about 900 μm; or wherein less than 5% of the granules have a diameter less than about 100 μm.

17. The process of claim 8, further comprising: compacting the mixture of step (b) followed by milling to form granules; and mixing of one or more of a flavour, saccharin sodium and a bicarbonate with the granules.

18. A process for the preparation of pharmaceutical composition of citric acid, sodium picosulfate, magnesium oxide, and a bicarbonate wherein the process comprises the steps of:
(a) mixing sodium picosulfate and citric acid;
(b) mixing magnesium oxide with the mixture of step (a);
(c) compacting the mixture of step (b) followed by milling to form granules; and
(d) mixing bicarbonate and optionally, one or both or flavours and sweeteners with the granules,
wherein the process does not involve use of any solvent.

19. The process of claim 18, wherein the process consists essentially of the steps of:
(a) mixing sodium picosulfate and citric acid;
(b) mixing magnesium oxide with the mixture of step (a);
(c) compacting the mixture of step (b) followed by milling to form granules; and
(d) mixing bicarbonate and optionally, one or both or flavours and sweeteners with the granules.

20. The process of claim 18, wherein the process consists of the steps of:
(a) mixing sodium picosulfate and citric acid;
(b) mixing magnesium oxide with the mixture of step (a);
(c) compacting the mixture of step (b) followed by milling to form granules; and
(d) mixing bicarbonate and, optionally, one or both or flavours and sweeteners with the granules.
